(19) United States
(12) Patent Application Publication
(10) Pub. No.: US 2026/0126440 A1
(43) Pub. Date: May 7, 2026

(54) METHODS FOR MEASURING LYMPHOCYTE ACTIVITY

(71) Applicant: Kite Pharma, Inc., Santa Monica, CA (US)

(72) Inventor: Chad M. Williams, Los Angeles, CA (US)

(21) Appl. No.: 19/375,588

(22) Filed: Oct. 31, 2025

Related U.S. Application Data
(60) Provisional application No. 63/716,067, filed on Nov. 4, 2024.

(51) Int. Cl. G01N 33/569

(52) U.S. Cl. CPC G01N 33/56972; G01N 2333/705; G01N 2333/70503; G01N 2333/70578

(57) ABSTRACT

Provided herein are methods for measuring antigen recognition response by lymphocytes upon exposure to a target antigen. Methods are disclosed for functionalizing red blood cells with antigens recognized by lymphocytes. By contacting antigen functionalized red blood cells with lymphocytes having specificity for the antigen, lymphocyte recognition response can be measured. The recognition response can be in the form of cytokine release by the lymphocytes and/or the expression of activation and/or differentiation markers on the surface of the lymphocytes.

### CROSS REFERENCE TO RELATED APPLICATIONS

This application claims the benefit of U.S. Provisional Ser. No. 63/716,067 , filed on Nov. 4, 2024, which is hereby incorporated by reference in its entirety.

### TECHNICAL FIELD

The present disclosure relates to the field of cell therapy, and more specifically, compositions and methods for measuring antigen recognition response in lymphocytes engineered to express a T-cell receptor and/or a chimeric antigen receptor.

### BACKGROUND

Cell therapy products prepared from lymphocytes have varying capacity for response to antigens, including but not limited to cytotoxic activity. One indicator of cytotoxic capacity of cell therapy products is cytokine release upon exposure to a target antigen. There is a need in the art for methods to characterize and quantify antigen recognition response by cell therapy products, e.g. cytokine release activity.

### SUMMARY

Described herein are methods and compositions for characterization and quantification of antigen recognition response by cell therapy products upon exposure to a target antigen, e.g. cytokine release. In certain aspects, cell therapy products are contacted with red blood cells which are functionalized with one or more target antigens specifically recognized by a T-cell receptor or chimeric antigen receptor that the lymphocytes of a cell therapy product are genetically engineered to express.

In accordance with a further embodiment of the present disclosure, therefore, provided is a method for measuring the antigen recognition response of a population of lymphocytes wherein at least one lymphocyte of the population of lymphocytes expresses a molecule having binding specificity for an antigen comprising: (a) affixing the antigen on a red blood cell (RBC) to create a functionalized RBC; (b) contacting the functionalized RBC with the population of lymphocytes; and (c) measuring for a response associated with antigen recognition exhibited by the population of lymphocytes, wherein the response is selected from the group consisting of (1) an increased cytokine release in comparison to a control population of the lymphocytes not contacted with the functionalized RBC; (2) the response is a greater proliferation of the population of lymphocytes in comparison to a control population of the lymphocytes not contacted with the functionalized RBC; (3) the response is an increased expression of cell surface activation markers of the population of lymphocytes in comparison to a control population of the lymphocytes not contacted with the functionalized RBC; (4) the response is an increased expression of cell surface differentiation markers of the population of lymphocytes in comparison to a control population of the lymphocytes not contacted with the functionalized RBC and any combination thereof.

In some embodiments, the population of lymphocytes comprises a cell type selected from the group consisting of a T cell, a B cell, a NK cell, a monocyte, a macrophage and any combination thereof. In some embodiments, the population of lymphocytes comprises a T cell.

In some embodiments, the population of lymphocytes comprises a cell expressing a chimeric antigen receptor (CAR).

In some embodiments, the population of lymphocytes comprises a cell expressing a T cell receptor.

In some embodiments, the antigen is affixed to the RBC by: 1) providing a RBC, 2) biotinylating the RBC, 3) coating the biotinylated RBC with streptavidin, and 4) contacting the streptavidin-coated RBC of step 3 with a biotinylated, avi-tagged antigen.

In some embodiments, the RBC is contacted with a biotin solution having a biotin concentration of 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 120 μM, 140 μM, 160 μM, 180 μM, 200 μM, 220 μM, 240 μM, 260 μM, 280 μM, 300 μM, 320 μM, 340 μM, 360 μM, 380 μM, 400 μM, 420 μM, 440μM, 460 μM, 480 μM, 500 μM, 520 μM, 540 μM, 560 μM, 580 μM, 600 μM, 620 μM, 640 μM, 660 μM, 680 μM, 700 μM, 720 μM, 740 μM, 760 μM, 780 μM, 800 μM, 820 μM, 840 μM, 860 M, 880 μM, 900 μM, 920 μM, 940 μM, 960 μM, 980 μM, or 1000 μM to provide for a biotinylated RBC.

In some embodiments, the biotinylated RBC is further contacted with a streptavidin solution having a streptavidin concentration of 1 mg/mL to provide for a biotin and streptavidin coated RBC.

In some embodiments, the biotin and streptavidin coated RBC is contacted with a biotinylated, avi-tagged antigen.

In some embodiments, the antigen is selected from the group consisting of DAP-10, CD 28, OX-40, 4-1BB (CD 137), CD 2,CD7,CD27,CD30,CD40, CD 70, OX40L, PD-L 1, CD 155 (PVR), 4-1BB ligand (4-1BBL), CD58, programmed death-1 (PD-1), inducible T cell costimulator (ICOS), lymphocyte function-associated antigen-1 (LFA-1, CD11a/CD18), CD3 gamma, CD3 delta, CD3 epsilon, CD247, CD276 (B7-H3), tumor necrosis factor superfamily member 14, TNFSF14, LIGHT), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, IA4, CD49D, ITGA6, VLA-6, CD49f, ICAM1, ITGAD (CD11d), ITGAE (CD103), ITGAL (CD11a), ITGAM (CD11b), ITGAX (CD11c), ITGB1, CD29, ITGB2, CD18, ITGB7, NKG2D, TNFR2, TRANCE (RANKL), DNAMI (CD226), SLAMF4 (CD244, 2B4), CD84, CD96 (Tactile), CEACAM1, CRTAM, Ly9(CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG (Cbp), CD19a, a ligand that specifically binds with CD83, ST4, alphafetoprotein (AFP), B-human chorionic gonadotropin, B7-1 (CD80), B7-2 (CD86), BCMA, CA-125, carcinoembryonic antigen (CEA), CD123, CD133, CD138, CD20, CD22, CD23, CD24, CD25, CD33, CD34, CD44, CD56, CD79b, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, GPC3, HBV-specific antigen, HCV-specific antigen, HERI-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFI)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, Nkp30, NY-ESO-1, p53, PAP, prostase, prostate specific antigen (PSA), prostate-carcinoma tumor antigen-1 (PCTA-1), prostate-specific antigen protein, PSMA, RAGE-1, ROR1, RUI, RU2 (AS), STEAP1, STEAP2, surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20) and any combinations thereof.

In some embodiments, the antigen is selected from the group consisting of CD19, CD20, CD22, CD33, CLL1, GPC3 and BCMA.

In some embodiments, the avi-tagged antigen is CD19.

In some embodiments, the antigen is a human CD19 protein fused to human serum albumin.

In some embodiments, the avi-tagged antigen is GPC3.

In some embodiments, the avi-tagged antigen is BCMA.

In some embodiments, the antigen comprises a combination of two or more antigens.

In some embodiments, the concentration of the antigen is selected to provide an average number of antigens affixed to an RBC between 100 antigen molecules per RBC to 500,000 antigen molecules per RBC.

In some embodiments, the number of molecules affixed to the individual functionalized RBC is up to 100, up to 200, up to 300, up to 400, up to 500, up to 600, up to 700, up to 800, up to 900, up to 1000, up to 2000, up to 3000, up to 4000, up to 5000, up to 6000, up to 7000, up to 8000, up to 9000, up to 10000, up to 15000, up to 20000, up to 25000, up to 30000, up to 35000, up to 40000, up to 45000, up to 50000, up to 75000, up to 100000, up to 125000, up to 150000, up to 175000, up to 200000, up to 225000, up to 250000, up to 275000, up to 300000, up to 325000, up to 350000, up to 375000, up to 400000, up to 425000, up to 450000, up to 475000, or up to 500000.

In some embodiments, the functionalized RBC and the population of lymphocytes are contacted at a predetermined ratio.

In some embodiments, the functionalized RBC to lymphocytes ratio is 1 to 100.

In some embodiments, the functionalized RBC to lymphocyte ratio is 1 to 10.

In some embodiments, the functionalized RBC to lymphocyte ratio is 1 to 1.

In some embodiments, the functionalized RBC to lymphocyte ratio is 10 to 1.

In some embodiments, the functionalized RBC and the population of lymphocytes are contacted for up to 12 hours.

In some embodiments, the functionalized RBC and the population of lymphocytes are contacted for up to 24 hours.

In some embodiments, the functionalized RBC and the population of lymphocytes are contacted for up to 36 hours.

In some embodiments, the functionalized RBC and the population of lymphocytes are contacted for up to 48 hours.

In some embodiments, the functionalized RBC and the population of lymphocytes are contacted for up to 3 days.

In some embodiments, the functionalized RBC and the population of lymphocytes are contacted for up to 4 days.

In some embodiments, the functionalized RBC and the population of lymphocytes are contacted for up to 5 days.

In some embodiments, the functionalized RBC and the population of lymphocytes are contacted for up to 6 days.

In some embodiments, the functionalized RBC and the population of lymphocytes are contacted for up to 7 days.

In some embodiments, the cytokine is selected from the group consisting of IL-2, IFNγ, Granzyme B, GM-CSF, TNFα, Perforin, IL-4, IL-5, IL-13, IL-17, IL-10, MIP-1a, MIP-1b, IL-6, sFasL, s4-1BB, Granzyme K and any combination thereof.

In some embodiments, the response is an increased cytokine release in comparison to a control population of the lymphocytes not contacted with the functionalized RBC.

In some embodiments, the response is a greater proliferation of the population of lymphocytes in comparison to a control population of the lymphocytes not contacted with the functionalized RBC.

In some embodiments, the response is an increased expression of cell surface activation markers of the population of lymphocytes in comparison to a control population of the lymphocytes not contacted with the functionalized RBC.

In some embodiments, the response is an increased expression of cell surface differentiation markers of the population of lymphocytes in comparison to a control population of the lymphocytes not contacted with the functionalized RBC.

In some embodiments, the cytokines are measured in supernatant removed from the mixture of the lymphocytes and functionalized RBCs.

In some embodiments, the response associated with antigen recognition is measured by flow cytometric analysis of the lymphocytes after the contacting of the lymphocytes with the functionalized RBCs.

In some embodiments, the method is performed in vitro.

In accordance with one embodiment of the present disclosure, therefore, provided is a method for measuring the cytokine release activity of a population of lymphocytes wherein at least one lymphocyte of the population of lymphocytes expresses a molecule having binding specificity for an antigen comprising: (a) affixing the antigen on a red blood cell (RBC) to create a functionalized RBC; (b)contacting the functionalized RBC with the population of lymphocytes; and (c) measuring the cytokines released by the population of lymphocytes.

### BRIEF DESCRIPTION OF THE DRAWINGS

FIGS. 1A-1B show antigen density as a function of biotin concentration. First, red blood cells (RBCs) were coated with biotin at various concentrations, then coated with streptavidin to saturation. The biotin/streptavidin coated RBCs were then contacted with avi-tagged, biotinylated CD19 antigen, and the CD19 antigen density on the RBCs was measured via flow cytometry and staining the RBCs with anti-human CD19 antibody. FIG. 1A shows the antigen expression of CD19 on the RBC surface with the x-axis representing anti-human CD19-PE antibody staining on the RBCs and y-axis showing cell counts normalized to the mode of each population. Histograms represent the CD19+ RBCs and the histogram at zero on the x-axis represents blank RBCs. FIG. 1B shows the concentration of biotin on the surface of the RBCs on the x-axis and CD19/molecules/cell on the y-axis. Dots represent the CD19+ RBCs populations and the number next to each dot represents the number of CD19 molecules/RBC. The graphs indicate that contacting the RBCs with increasing biotin concentration resulted in a higher density of CD19 antigen molecules on the functionalized RBCs.

FIGS. 2A-2C show effector cell (anti-CD19 CAR-T cell) cytokine production as a function of varying the ratio of functionalized RBCs (RBCs displaying CD19 antigen) to effector cell at the contacting step and CD19 expression on the RBC. Functionalized RBCs coated with different densities of CD19 antigen were contacted with effector cells that specifically bind to the antigen affixed to the RBCs. Upon contacting, the IL-2, IFNγ, and granzyme B cytokines released by the effector cells were measured. FIG. 2A shows RBC: Effector cell ratio along x-axis and concentration of IL-2 (ng/mL) in y-axis. The legend identifies the different populations of RBCs with different surface expression of CD19 molecules per RBC. FIG. 2B shows RBC: Effector cell ratio along x-axis and concentration of IFNγ (ng/ml) in y-axis. Again, the legend identifies the different populations of RBCs with different surface expression of CD19 molecules per RBC. FIG. 2C shows RBC: Effector cell ratio along x-axis and concentration of Granzyme B (ng/ml) in y-axis. The legend identifies the different populations of RBCs with different surface expression of CD19 molecules per RBC. The graphs indicate that various ratios of functionalized RBCs to effector cells at the contacting step result in varying amount of cytokine production, and that the expression of CD19 on the surface of the RBC also determines the maximum amount of cytokine production.

FIGS. 3A-3E show the concentration of cytokines released by effector cells (anti-CD19 CAR-T cells) upon contacting with CD19 antigen displaying RBCs, varies based on the density of antigens functionalized to RBCs. Anti-CD19 CAR T-cells were contacted with RBCs functionalized with CD19 antigen at a 1:1 ratio, and the cytokines released by the anti-CD 19 CAR-T cells were measured. Generally, the concentration of cytokines secreted by the anti-CD19 CAR T-cells increased with increasing density of CD19 on the RBCs. FIG. 3A shows CD19 expression on the surface of the RBC (CD19 molecules/cell) on x-axis vs. IFNγ cytokine production (ng/ml) on y-axis. FIG. 3B shows CD19 expression on the surface of the RBC (CD19 molecules/cell) on x-axis vs. IL-2 cytokine production (ng/ml) on y-axis. FIG. 3C shows CD19 expression on the surface of the RBC (CD19 molecules/cell) on x-axis vs. IL-4 cytokine production (ng/ml) on y-axis. FIG. 3D shows CD19 expression on the surface of the RBC (CD19 molecules/cell) on x-axis vs. IL-5 cytokine production (ng/ml) on y-axis. FIG. 3E shows CD19 expression on the surface of the RBC (CD19 molecules/cell) on x-axis vs. IL-13 cytokine production (ng/ml) on y-axis. Legend shows subject ID (de-identified) of product cells from patients on Zuma-1 trial. Dotted line shows 3000 CD19 molecules/RBC.

FIGS. 4A-4C show that anti-CD19 CAR T-cells secrete increasing levels of the cytokine IFNγ if contacted with increasing amounts of CD19 molecules per RBC and at different effector to RBC ratios in the well. CD19 molecules per well was calculated based on number of CD19 antigen displaying RBC per well multiplied by the number of CD19 antigen molecules expressed per RBC (ex. #RBC/well×#CD19/RBC). Generally, the antigen density (#CD19/RBC) is more influential on IFNγ cytokine secretion than the ratio of effector cell to RBC in the well. FIG. 4A shows greater IFNγ release as a function of antigen present in the well. FIG. 4B shows greater IFNγ release as a function of antigen density on the functionalized RBC. FIG. 4C shows IFNγ release as a function of RBC: T cell ratio changes.

FIGS. 5A-5B show flow data representing activation marker 4-1BB on either CD4 or CD8 CAR T cells compared to cytokine production of IL-2 and IFNγ due to stimulation with CD19 antigen displaying RBCs. T helper, CD4 T cells are known to maintain proliferation and there was a strong relationship observed between the presence of activated 4-1BB+CD4+ CAR T cells and the T cell proliferation inducing cytokine, IL-2, after effector cell stimulation with CD19antigen displaying RBCs. FIG. 5A shows that RBCs functionalized with a higher antigen density stimulated greater release of IL-2 at a given abundance of activated 4-1BB+CD4+ CAR T cells. FIG. 5B shows that IFNγ release was dependent on the antigen density of CD19 on functionalized RBCs and has a trend with the abundance of activated 4-1BB+CD8+CAR T cells. While pro-inflammatory cytokine IFNγ was more strongly related to the abundance of activated 4-1BB+CD8+ CAR T cells after stimulation, T cell proliferation inducing cytokine, IL-2, was more strongly related to activated 4-1BB+CD4+ CAR T cells.

FIG. 6 shows the stability of CD19 expression on CD19 antigen displaying RBCs over time. Various densities of CD19 were conjugated to the surface of RBCs, and expression was monitored over the period of 36 days. X-axis shows the days after CD19 protein coating of the RBC, and y-axis shows the CD19 molecules per RBC. Legend denotes the concentration of biotin contacted on RBC surface to generate the CD19 expression quantified for the experiment. Different colors represent different populations of RBC. In general, over the course of roughly one month there was little variation in the CD19 expression on CD19 antigen displaying RBCs. This allows for ease of use of these CD19 antigen displaying RBCs, in that it is not required to make RBCs fresh before every experiment and CD19 antigen expression is stably maintained on the RBC surface over time.

FIGS. 7A-7C show antigen density as a function of biotin concentration. First, red blood cells (RBCs) were coated with biotin at various concentrations, then coated with streptavidin to saturation. The biotin/streptavidin coated RBCs were then contacted with avi-tagged, biotinylated GPC3 antigen, and the GPC3 antigen density on the RBCs was measured via flow cytometry and staining the RBCs with anti-human GPC3 antibody. FIG. 7A shows the antigen expression of GPC3 on the RBC surface with the x-axis representing anti-human GPC3-PE antibody staining on the RBCs and y-axis showing cell counts normalized to the mode of each population. The histograms represent the GPC3+ RBCs and the histogram at zero on the x-axis represents blank RBCs. FIG. 7B shows that contacting the RBCs with increasing biotin concentration resulted in a higher density of GPC3 antigen molecules on the functionalized RBCs. X-axis represents biotin concentration contacted on RBC, and y-axis shows the GPC3 molecules per RBC. FIG. 7C shows the functionalized GPC3 RBCs co-cultured with effector cells increase the frequency of activation marker CD69 expressed by effector cells (anti-GPC3 CAR-T cells) upon contacting with GPC3 antigen displaying RBCs, varies based on the density of antigens functionalized to RBCs in the well. X-axis shows negative controls T cell alone, irrelevant protein expressing RBCs (CD19), and then antigen density of GPC3 molecules per RBC. Three different anti-GPC3 CAR T-cells were contacted with RBCs functionalized with GPC3 antigen, and the CD69 expressed by the anti-GPC3 CAR-T cells were measured. Generally, the CD69 expressed by the anti-GPC3 CAR T-cells increased with increasing density of GPC3 on the RBCs.

FIGS. 8A-8D show the BCMA antigen density on the RBCs was measured via flow cytometry by staining the RBCs with anti-human BCMA antibody. FIG. 8A indicates 6 different BCMA densities of BCMA antigen molecules on the functionalized RBCs and the histogram at zero on the x-axis represents blank RBCs, with the x-axis representing anti-human BCMA-PE antibody staining on the RBCs and y-axis showing cell counts normalized to the mode of each population. FIG. 8B shows that contacting the RBCs with increasing biotin concentration resulted in a higher density of BCMA antigen molecules on the functionalized RBCs. X-axis represents biotin concentration contacted on RBC, and y-axis shows the BCMA molecules per RBC. FIG. 8C shows the functionalized BCMA RBCs co-cultured with effector cells increase the frequency of activation marker CD69 expressed by effector cells (anti-GPC3 CAR-T cells) upon contacting with BCMA antigen displaying RBCs, varies based on the density of antigens functionalized to RBCs in the well. X-axis shows negative control irrelevant protein expressing RBCs (CD19), and then antigen density of BCMA molecules per RBC. FIG. 8D depicts functionalized BCMA RBCs co-cultured with effector cells showed the concentration of cytokines released by effector cells (anti-BCMA CAR-T cells) upon contacting with BCMA antigen displaying RBCs, varies based on the density of antigens functionalized to RBCs in the well. Three different anti-BCMA CAR T-cells were contacted with RBCs functionalized with BCMA antigen, and the cytokines released by the anti-BCMA CAR-T cells were measured. Generally, the concentration of cytokines secreted by the anti-BCMA CAR T-cells increased with increasing density of BCMA on the RBCs.

FIG. 9A shows a schematic of how the presently disclosed assay method can be performed for two antigens, for example CD19 and a co-stimulatory ligand. As shown in FIG. 9A, CD19 avi-tag solution was added to RBCs for 15 minutes and then co-stimulatory ligand solution was added for the final 15 minutes of incubation (30 min. incubation total for CD19). FIG. 9B shows flow-based quantification with the y-axis showing cell counts normalized to the mode of each population and the x-axis representing anti-human CD19-PE, anti-human OX40L-PE, or anti-human ICAM-1—PE antibody staining on the RBCs. FIG. 9B shows that both CD19and co-stimulatory ligand expression in Dual Coated RBCs have similar uniformity and antigen distribution. In addition, CD19 antigen density on single coated CD19 is similar to CD19 antigen density on Dual Coated CD19 RBCs. FIG. 9C shows that CD19 effector cell cytokine production was quantified for single and dual coated CD19 RBC stimulation and ratio between cytokine production with CD19 alone or CD19 with co-stimulatory ligand expression was calculated (>1=increase in cytokine production compared to CD19 alone, <1=decrease in cytokine production compared to CD19 alone). Addition of PD-L1 was shown to decrease cytokine production, while addition of 4-1BBL increased cytokine production.

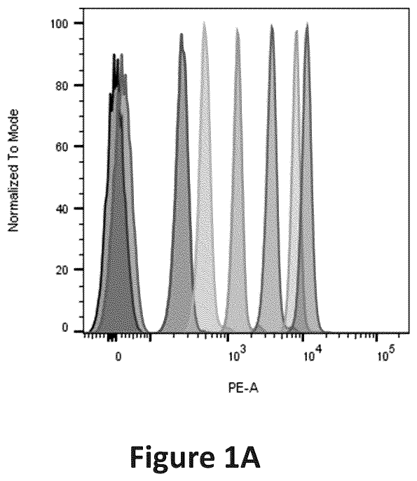

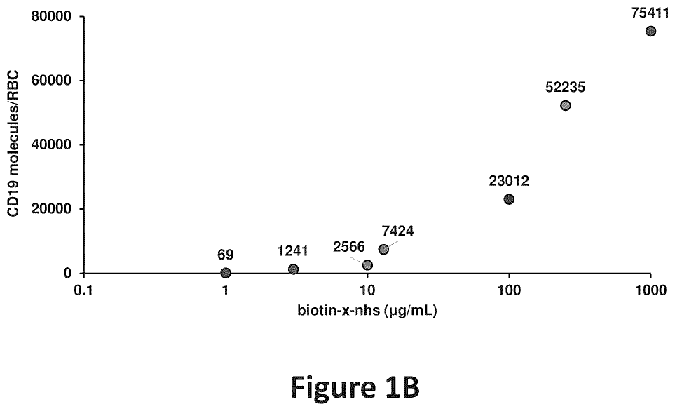

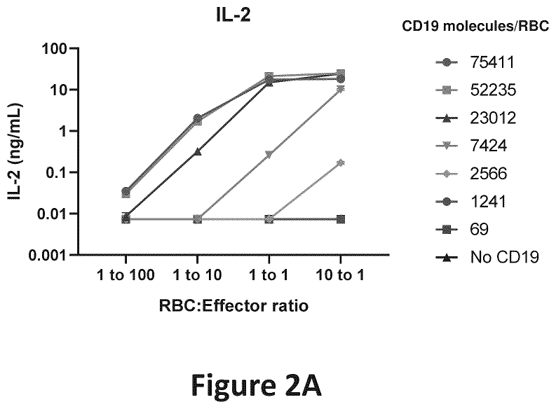

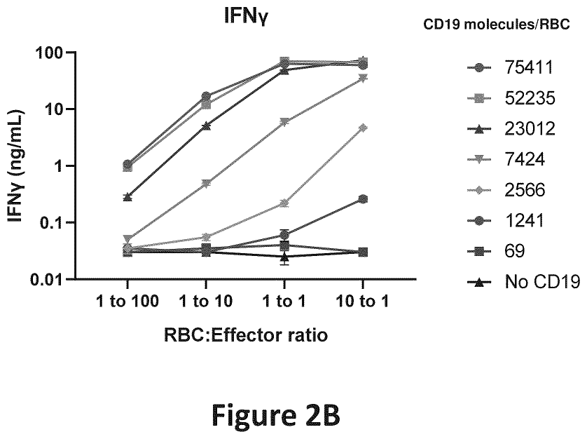

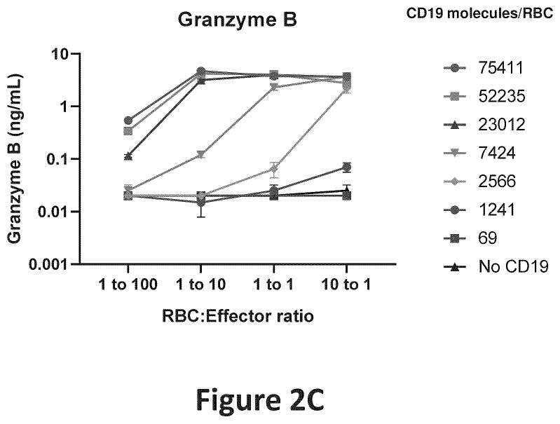

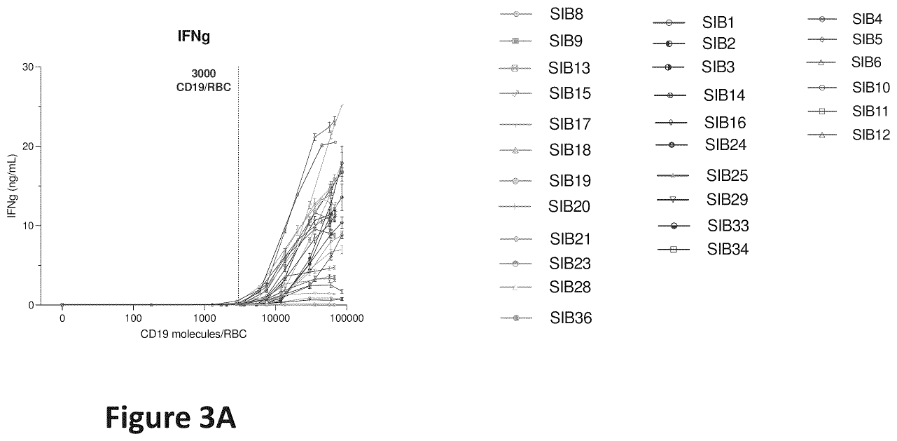

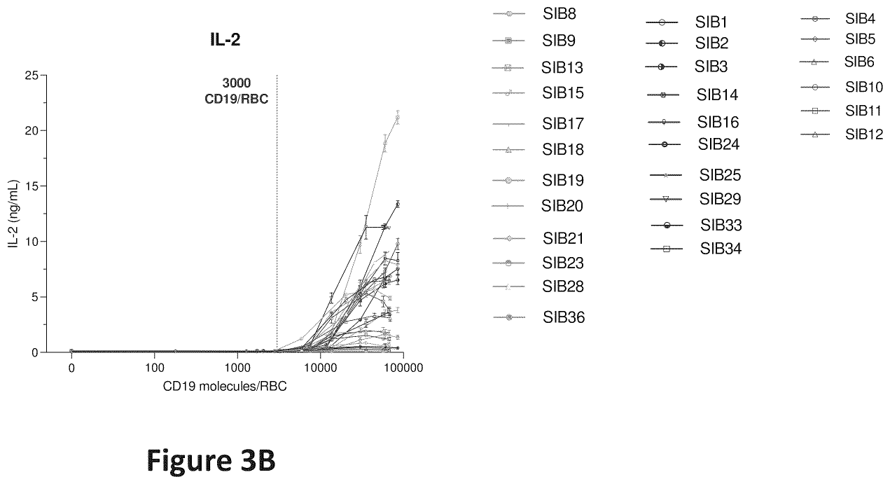

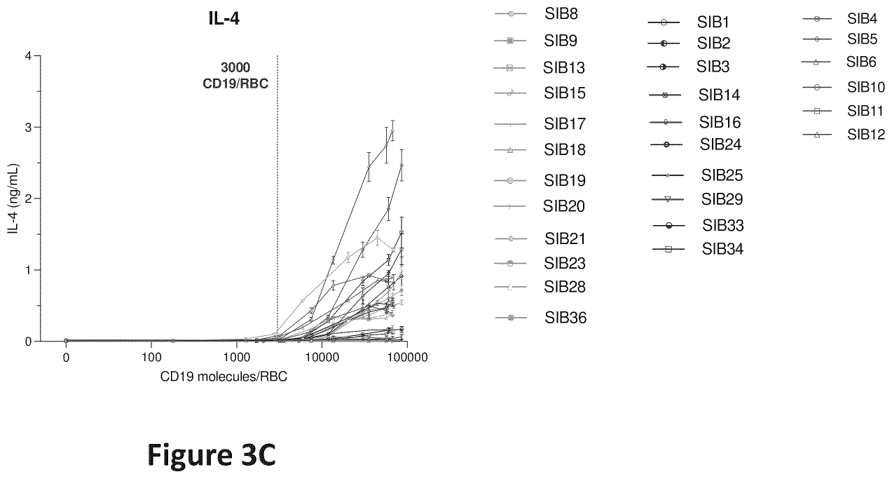

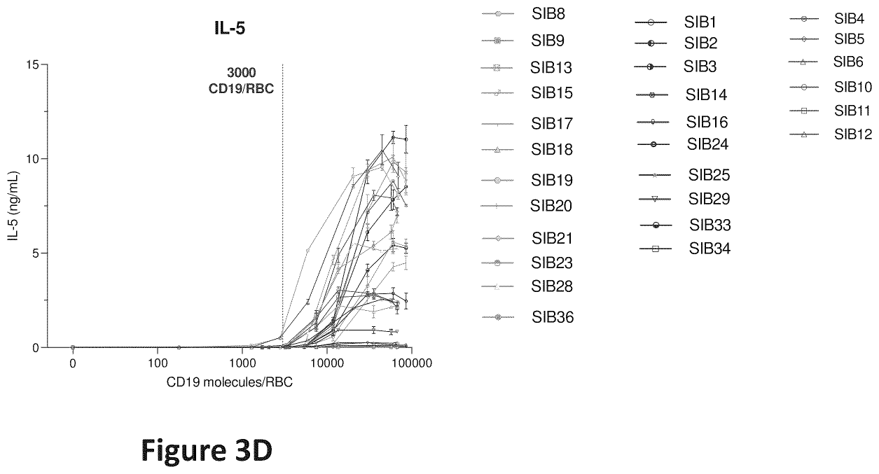

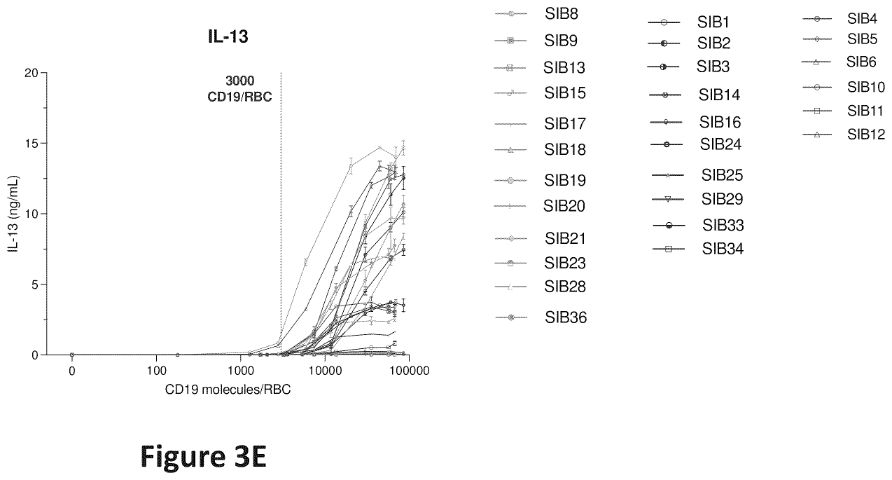

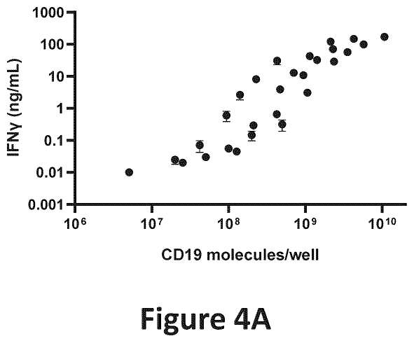

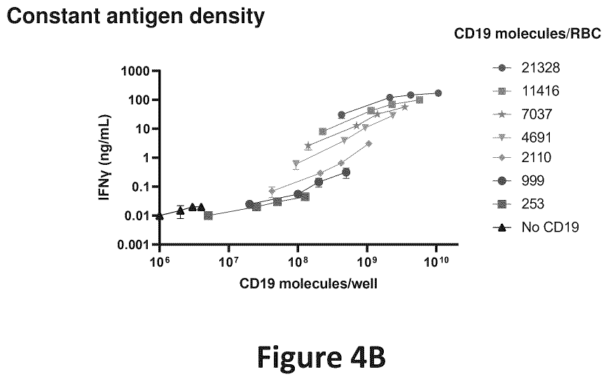

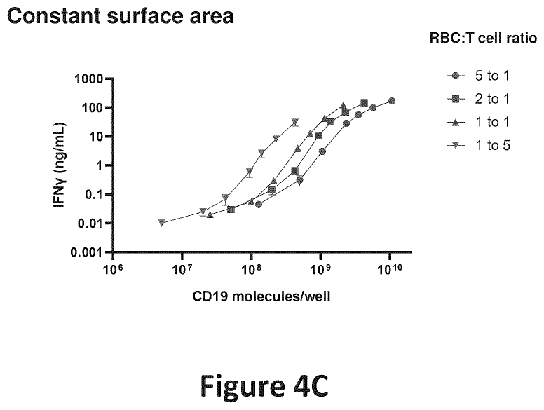

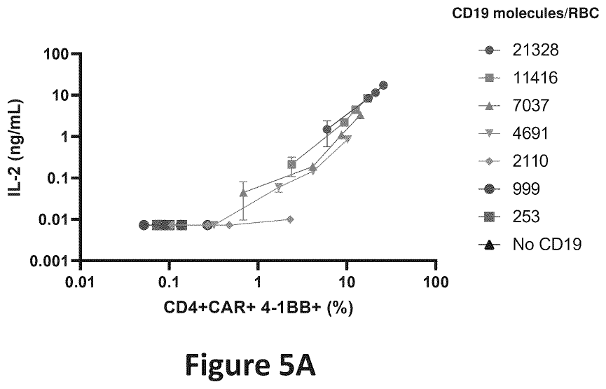

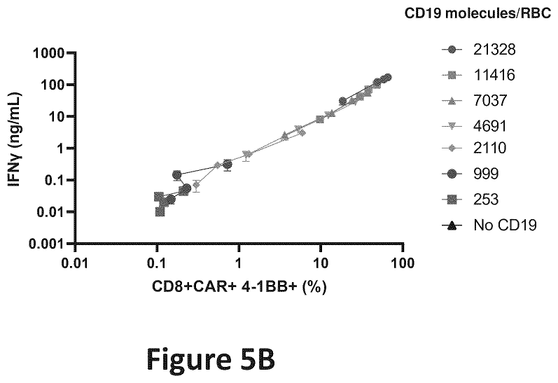

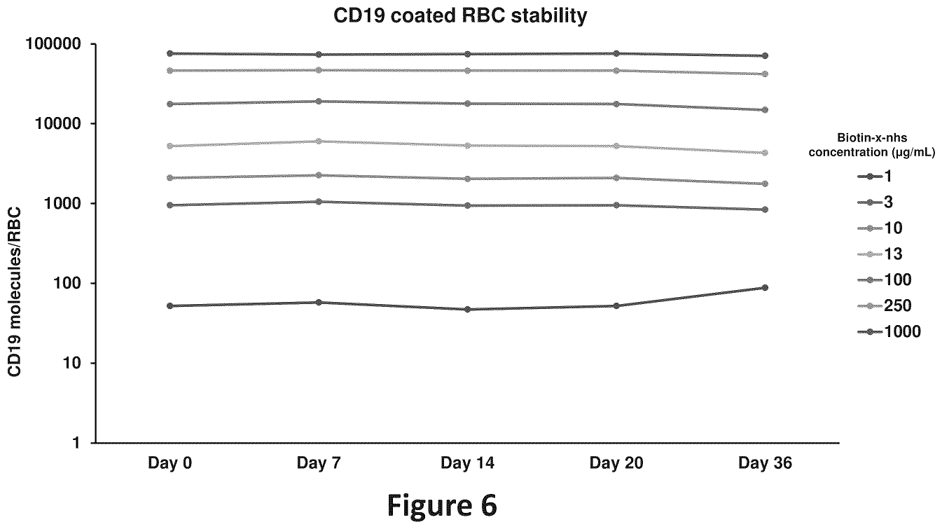

### DETAILED DESCRIPTION

### Definitions

Except as otherwise expressly provided herein, each of the following terms shall have the meaning set forth below. Additional definitions are set forth throughout the application. Unless defined otherwise, all technical and scientific terms used herein have the meaning as commonly understood by one of ordinary skill in the art. For example, the manual Current Protocols In Immunology, edited by John E. Coligan, Ada M. Kruisbeek, David H. Margulies, Ethan M. Shevach, Warren Strober, (Series Editior: Richard Coico), ISBN 0471522767; Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2006, Oxford University Press, provide one of skill with a general dictionary of many of the terms used in this application.

Units, prefixes, and symbols are denoted in their Système International de Unites (SI) accepted form. Numeric ranges are inclusive of the numbers defining the range. The disclosure provided herein are not limitations of the various aspects of the application, which may be by reference to the specification as a whole. Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

The articles “a” or “an” refer to “one or more” of any recited or enumerated component.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., +10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The terms “e.g.,” and “i.e.,” are used merely by way of example, without limitation intended, and not be construed as referring only those items explicitly enumerated in the specification.

The terms “or more”, “at least”, “more than”, and the like, e.g., “at least one” include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes each value less than the stated value. For example, “no more than xyx” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 xyz. Also included is any lesser number or fraction in between.

The terms “plurality”, “at least two”, “two or more”, “at least second”, and the like include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

The term “allogeneic” refers to any material derived from one individual which is then introduced to another individual of the same species, e.g., allogeneic T cell transplantation.

The term “autologous” refers to a therapeutic intervention that uses an individual's own cells or tissues, which are processed outside the body, and reintroduced into the individual.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The “antigen” may be the tumor antigen selected from 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2,-3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), k-light chain, LAGE (L antigen), LDLR/FUT (low density lipid receptor/GDP-L-fucose:
b-D-galactosidase 2-a-Lfucosyltransferase), LeY (Lewis-Y antibody), L1CAM (L1 cell adhesion molecule), MAGE (melanoma antigen), MAGE-A1 (Melanoma-associated antigen 1), MAGE-A3, MAGE-A6, mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1,-2,-3 (melanoma ubiquitous mutated 1, 2, 3), NA 88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1,-2,-3,-4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1(tyrosinase related protein 1, or gp75), TRP-2 (tyrosinase related protein 2), TRP-2/INT2 (TRP-2/intron 2), VEGF-R2 (vascular endothelial growth factor receptor 2), WT1 (Wilms'tumor gene), and any combination thereof. In one embodiment, the tumor antigen is CD19.

In one embodiment, the T cell products of the disclosure are used in “CD19-directed genetically modified autologous T cell immunotherapy,”which refers to a suspension of chimeric antigen receptor (CAR)-positive immune cells. An example of such immunotherapy is Clear CAR-T therapy, which uses CAR-T cells that are free of circulating tumor cells and enriched in CD4+/CD8+T cells. Another example is axicabtagene ciloleucel (also known as Axi-cel™, YESCARTA®). See Kochenderfer, et al., (J Immunother 2009; 32:689 702). In one embodiment, the T cell product is brexucabtagene autoleucel (formerly KTE-X19; Tecartus) Other non-limiting examples include JCAR017, JCAR015, JCAR014, Kymriah (tisagenlecleucel), Uppsala U. anti-CD19 CAR (NCT02132624), and UCART19 (Celectis). See Sadelain et al. Nature Rev. Cancer Vol. 3(2003 ), Ruella et al., Curr Hematol Malig Rep., Springer, NY (2016) and Sadelain et al. Cancer Discovery (Apr 2013) To prepare CD 19-directed genetically modified autologous T cell immunotherapy, a patient's own T cells may be harvested and genetically modified ex vivo by retroviral transduction to express a chimeric antigen receptor (CAR) comprising a murine anti-CD19 single chain variable fragment (scFv) linked to CD28 and CD3-zeta co-stimulatory domains. In some embodiments, the CAR comprises a murine anti-CD19 single chain variable fragment (scFv) linked to 4-1BB and CD 3-zeta co-stimulatory domain. The anti-CD 19 CAR T cells may be expanded and infused back into the patient, where they may recognize and eliminate CD19-expressing target cells.

In some embodiments, the T cells are engineered with a T cell receptor (TCR), which may comprise a binding molecule to a tumor antigen. In some aspects, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1,CASP-8,CDC27m, CDK4/m, CEA, CT, Cyp-B, DAM, EGFR, EGFRVIII, EGP-2, EGP-40, Erbb2, 3, 4, ELF2M, ETV6-AML1, FBP, fAchR, G250, GAGE, GD2, GD3, GnT-V, Gp100, HAGE, HER-2/neu, HLA-A, HPV, HSP70-2M, HST-2, hTERT or hTRT, iCE, IL-13R-a2, KIAA0205, KDR, κ-light chain, LAGE, LDLR/FUT, LeY, LICAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen, P15, p190 minor bcr-abl, Pml/RARa, PRAME, PSA, PSCA, PSMA, RAGE, RU1 or RU2, SAGE, SART-1 or SART-3, SSX1,-2,-3, 4, TAA, TAG-72, TEL/AML1, TPI/m, TRP-1, TRP-2, TRP-2/INT2, VEGF-R2, WT1, and any combination thereof. In one aspect, the TCR comprises a binding molecule to a viral oncogene. In one embodiment, the viral oncogene is selected from human papilloma virus (HPV), Epstein-Barr virus (EBV), and human T-lymphotropic virus (HTLV). In other embodiments, the TCR comprises a binding molecule to a testicular, placental, or fetal tumor antigen. In one embodiment, the testicular, placental, or fetal tumor antigen is selected from the group consisting of NY-ESO-1, synovial sarcoma X breakpoint 2 (SSX2), melanoma antigen (MAGE), and any combination thereof. In another embodiment, the TCR comprises a binding molecule to a lineage specific antigen. In additional embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof. In certain embodiment, the T cell therapy comprises administering to the patient engineered CAR T cells expressing a chimeric antigen receptor that binds to CD19 and further comprises a CD28 costimulatory domain and a CD3-zeta signaling region. In additional embodiment, the T cell therapy comprises administering to a patient KTE-C19 or KTE-X19. In one aspect, the antigenic moieties also include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, I, gJ, gK, gL, gM, UL20, UL32, US43, UL45, UL49A), a cytomegalovirus (CMV) viral antigen (e.g., glycoproteins gB, gC, gC, gE, gG, gH, gI, gJ, gK, gL. gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such aspects, the cell surface receptor may be any TCR, or any CAR which recognizes any of the aforementioned viral antigens on a target virally infected cell. In other aspects, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor at various stages. In one embodiment, the cancer or tumor is stage 0, such that, e.g., the cancer or tumor is very early in development and has not metastasized. In another embodiment, the cancer or tumor is stage I, such that, e.g., the cancer or tumor is relatively small in size, has not spread into nearby tissue, and has not metastasized. In other embodiment, the cancer or tumor is stage II or stage III, such that, e.g., the cancer or tumor is larger than in stage 0 or stage I, and it has grown into neighboring tissues but it has not metastasized, except potentially to the lymph nodes. In additional embodiment, the cancer or tumor is stage IV, such that, e.g., the cancer or tumor has metastasized. Stage IV may also be referred to as advanced or metastatic cancer.

In certain embodiments, the cancer may be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sèzary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias may affect one or more of these cell types in a patient.

In general, lymphomas may be divided into at least two sub-groups: Hodgkin lymphoma and non-Hodgkin lymphoma. Non-Hodgkin Lymphoma (NHL) is a heterogeneous group of cancers originating in B lymphocytes, T lymphocytes or natural killer cells. In the United States, B cell lymphomas represent 80-85% of cases reported. In 2013 approximately 69,740 new cases of NHL and over 19,000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer.

Examples of B cell malignancies include, but are not limited to, Non-Hodgkin's Lymphomas (NHL), Small lymphocytic lymphoma (SLL/CLL), Mantle cell lymphoma (MCL), FL, Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some aspects, the lymphoma or leukemia is selected from B-cell chronic lymphocytic leukemia/small cell lymphoma, B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some aspect, the cancer is refractory to one or more prior treatments, and/or the cancer has relapsed after one or more prior treatments.

In one embodiment, the cancer is selected from follicular lymphoma, transformed follicular lymphoma, diffuse large B cell lymphoma, and primary mediastinal (thymic) large B-cell lymphoma. In another embodiment, the cancer is diffuse large B cell lymphoma. In some embodiment, the cancer is refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In one embodiment, the cancer is refractory diffuse large B cell lymphoma or mantle cell lymphoma.

The term “lymphocyte” as used herein may include natural killer (NK) cells, T cells, NK-T cells, or B cells. NK cells are a type of cytotoxic (cell toxic) lymphocyte that represent a major component of the inherent immune system. NK cells reject tumors and cells infected by viruses, through the process of apoptosis or programmed cell death. They were termed “natural killers” because they do not require activation to kill cells. T-cells play a major role in cell-mediated immunity (no antibody involvement). The T-cell receptors (TCR) differentiate themselves from other lymphocyte types. The thymus, a specialized organ of the immune system, is primarily responsible for the T cell's maturation.

There are several types of “immune cells,” including, without limitation, macrophages (e.g., tumor associated macrophages) neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells. The term also includes precursors of these immune cells. Hematopoietic stem and/or progenitor cells may be derived from bone marrow, umbilical cord blood, adult peripheral blood after cytokine mobilization, and the like, by methods known in the art. Some precursor cells are those that may differentiate into the lymphoid lineage, for example, hematopoietic stem cells or progenitor cells of the lymphoid lineage. Additional examples of immune cells that may be used for immune therapy are described in US Publication No. 20180273601, incorporated herein by reference in its entirety.

There are also several types of T-cells, namely: Helper T-cells (e.g., CD4+ cells, effector TEFF cells), Cytotoxic T-cells (also known as TC, cytotoxic T lymphocyte, CTL, T-killer cell, cytolytic T cell, CD8+T-cells or killer T cell), Memory T-cells ((i) stem memory T_{SCM} cells, like naive cells, are CD45RO−, CCR7+, CD45RA+, CD62L+(L-selectin), CD27+, CD28+and IL-7Ra+, but they also express large amounts of CD95, IL-2R$, CXCR3, and LFA-1, and show numerous functional attributes distinctive of memory cells); (ii) central memory T_{CM} cells express L-selectin and are CCR7⁺and CD45RO⁺ and they secrete IL-2, but not IFNγ or IL-4, and (iii) effector memory T_{EM} cells, however, do not express L-selectin or CCR7 but do express CD45RO and produce effector cytokines like IFNγ and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” (TIL). B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

A “naïve” T cell refers to a mature T cell that remains immunologically undifferentiated. Following positive and negative selection in the thymus, T cells emerge as either CD4⁺ or CD8⁺ naïve T cells. In their naïve state, T cells express L-selectin (CD62L⁺), IL-7 receptor-α (IL-7R-α), and CD132, but they do not express CD25, CD44, CD69, or CD45RO. As used herein, “immature” may also refer to a T cell which exhibits a phenotype characteristic of either a naïve T cell or an immature T cell, such as a T_{SCM} cell or a T_{CM} cell. For example, an immature T cell may express one or more of L-selectin (CD62L⁺), IL-7Rα, CD132, CCR7, CD45RA, CD45RO, CD27, CD28, CD95, IL-2Rβ, CXCR3, and LFA-1. Naïve or immature T cells may be contrasted with terminal differentiated effector T cells, such as T_{EM} cells and T_{EFF} cells.

The terms cell “proliferation,” “proliferating” or the like refer to the ability of cells to grow in numbers through cell division. Proliferation may be measured by staining cells with carboxyfluorescein succinimidyl ester (CFSE). Cell proliferation may occur in vitro, e.g., during T cell culture, or in vivo, e.g., following administration of a immune cell therapy (e.g., T cell therapy). The cell proliferation may be measured or determined by the methods described herein or known in the field. For example, cell proliferation may be measured or determined by viable cell density (VCD) or total viable cell (TVC). VCD or TVC may be theoretical (an aliquot or sample is removed from a culture at certain timepoint to determine the cell number, then the cell number multiples with the culture volume at the beginning of the study) or actual (an aliquot or sample is removed from a culture at certain timepoint to determine the cell number, then the cell number multiples with the actual culture volume at the certain timepoint). The term “T cell activity” refers to any activity common to healthy T cells. In one embodiment, the T cell activity comprises cytokine production (such as INFγ, IL-2, and/or TNFα). In other embodiment, the T cell activity comprises production of one or more cytokine selected from interferon gamma (IFNγ or IFN-γ), tissue necrosis factor alpha (TNFα or IFNα), and both. The terms “cytolytic activity,” “cytotoxicity” or the like refer to the ability of a T cell to destroy a target cell. In one embodiment, the target cell is a cancer cell, e.g., a tumor cell. In other embodiments, the T cell expresses a chimeric antigen receptor (CAR) or a T cell receptor (TCR), and the target cell expresses a target antigen.

The term “genetically engineered,” “gene editing,” or “engineered” refers to a method of modifying the genome of a cell, including, but not being limited to, deleting a coding or non-coding region or a portion thereof or inserting a coding region or a portion thereof. In one embodiment, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

The terms “transduction” and “transduced” refer to the process whereby foreign DNA is introduced into a cell via viral vector (see Jones et al., “Genetics: principles and analysis,” Boston:
Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR-T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, α, β, γ, and δ, may rearrange leading to highly diverse TCR proteins. In some embodiments, the T cells are engineered with a T cell receptor (TCR), which may comprise a binding molecule to a tumor antigen. In some aspects, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1, CASP-8, CDC27m, CDK4/m, CEA, CT, Cyp-B, DAM, EGFR, EGFRvIII, EGP-2, EGP-40, Erbb2, 3, 4, ELF2M, ETV6-AML1, FBP, fAchR, G250, GAGE, GD2, GD3, GnT-V, Gp100, HAGE, HER-2/neu, HLA-A, HPV, HSP70-2M, HST-2, hTERT or hTRT, iCE, IL-13R-a2, KIAA0205, KDR, K-light chain, LAGE, LDLR/FUT, LeY, LICAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen, P15, p190 minor bcr-abl, Pml/RARa, PRAME, PSA, PSCA, PSMA, RAGE, RU1 or RU2, SAGE, SART-1 or SART-3, SSX1, -2, -3, 4, TAA, TAG-72, TEL/AML1, TPI/m, TRP-1, TRP-2, TRP-2/INT2, VEGF-R2, WT1, and any combination thereof. In one aspect, the TCR comprises a binding molecule to a viral oncogene. In one embodiment, the viral oncogene is selected from human papilloma virus (HPV), Epstein-Barr virus (EBV), and human T-lymphotropic virus (HTLV). In other embodiments, the TCR comprises a binding molecule to a testicular, placental, or fetal tumor antigen. In one embodiment, the testicular, placental, or fetal tumor antigen is selected from the group consisting of NY-ESO-1, synovial sarcoma X breakpoint 2 (SSX2), melanoma antigen (MAGE), and any combination thereof. In another embodiment, the TCR comprises a binding molecule to a lineage specific antigen. In additional embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof. In certain embodiment, the T cell therapy comprises administering to the patient engineered CAR T cells expressing a chimeric antigen receptor that binds to CD19 and further comprises a CD28 costimulatory domain and a CD3-zeta signaling region. In additional embodiment, the T cell therapy comprises administering to a patient KTE-C19 or KTE-X19. In one aspect, the antigenic moieties also include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL, gM, UL20, UL32, US43, UL45, UL49A), a cytomegalovirus (CMV) viral antigen (e.g., glycoproteins gB, gC, gC, gE, gG, gH, gI, gJ, gK, gL, gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such aspects, the cell surface receptor may be any TCR, or any CAR which recognizes any of the aforementioned viral antigens on a target virally infected cell. In other aspects, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation, OPTIPREP™ separation, and/or apheresis. Additional methods of isolating immune cells for an immune cell therapy are disclosed in U.S. Patent Publication No. 2013/0287748, which is herein incorporated by reference in its entirety.

A “patient” as used herein includes any human who is afflicted with a disease or disorder, including cancer (e.g., a lymphoma or a leukemia). The terms “subject” and “patient” are used interchangeably herein. The term “donor subject” refers to herein a subject whose cells are being obtained for further in vitro engineering. The donor subject may be a cancer patient that is to be treated with a population of cells generated by the methods described herein (i.e., an autologous donor), or may be an individual who donates a lymphocyte sample that, upon generation of the population of cells generated by the methods described herein, will be used to treat a different individual or cancer patient (i.e., an allogeneic donor). Those subjects who receive the cells that were prepared by the present methods may be referred to as “recipient subject.”

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre-and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR-T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign nucleic acid is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

The one or more immune cells described herein may be obtained from any source, including, for example, a human donor. The donor may be a subject in need of an anti-cancer treatment, e.g., treatment with one immune cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). immune cells may be differentiated in vitro from a hematopoietic stem cell population, or immune cells may be obtained from a donor. The population of immune cells may be obtained from the donor by any suitable method used in the art. For example, the population of lymphocytes may be obtained by any suitable extracorporeal method, venipuncture, or other blood collection method by which a sample of blood with or without lymphocytes is obtained. The population of lymphocytes is obtained by apheresis. The one or more immune cells may be collected from any tissue that comprises one or more immune cells, including, but not limited to, a tumor. A tumor or a portion thereof is collected from a subject, and one or more immune cells are isolated from the tumor tissue. Any T cell may be used in the methods disclosed herein, including any immune cells suitable for a T cell therapy. For example, the one or more cells useful for the application may be selected from the group consisting of tumor infiltrating lymphocytes (TIL), cytotoxic T cells, CAR T cells, engineered TCR T cells, natural killer T cells, Dendritic cells, and peripheral blood lymphocytes. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. T cells may also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety. In one embodiment, T cells are tumor infiltrating leukocytes. In certain embodiment, the one or more T cells express CD8, e.g., are CD8⁺ T cells. In other embodiment, the one or more T cells express CD4, e.g., are CD4⁺ T cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entiretyIn some aspect, the cells of the present application may be obtained through T cells obtained from a subject. In one aspect, the T cells may be obtained from, e.g., peripheral blood mononuclear cells (PBMC), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In some aspect, the cells collected by apheresis are washed to remove the plasma fraction and placed in an appropriate buffer or media for subsequent processing. In some aspect, the cells are washed with any solution (e.g., a solution with neutralized PH value or PBS) or culture medium. As will be appreciated, a washing step may be used, such as by using a semiautomated flow through centrifuge, e.g., the Cobe™ 2991 cell processor, the Baxter CytoMate™, or the like. In some aspect, the washed cells are resuspended in one or more biocompatible buffers, or other saline solution with or without buffer. In some aspect, the undesired components of the apheresis sample are removed. Additional methods of isolating T cells for a T cell therapy are disclosed in U.S. Patent Pub. No. 2013/0287748, which are hereby incorporated by references in their entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In one embodiment, CD3+T cells are isolated from PBMCs using Dynabeads coated with anti-CD3 antibody. CD8+ and CD4+ T cells are further separately isolated by positive selection using CD8 microbeads (e.g., Miltenyi Biotec) and/or CD4 microbeads (e.g., Miltenyi Biotec).

PBMCs may be used directly for genetic modification with the immune cells (such as CARs). After isolating the PBMCs, T lymphocytes are further isolated, and both cytotoxic and helper T lymphocytes are sorted into naive, memory, and effector T cell subpopulations either before or after genetic modification and/or expansion. In one embodiment, CD8+ cells may be further sorted into naive, central memory, and effector cells by identifying cell surface antigens that are associated with each of these types of CD8+ cells. In other embodiment, the expression of phenotypic markers of central memory T cells includes CCR7, CD3, CD28, CD45RO, CD62L, and CD127 and are negative for granzyme B. In some embodiment, central memory T cells are CD8+, CD45RO+, and CD62L+ T cells. In certain embodiment, effector T cells are negative for CCR7, CD28, CD62L, and CD127 and positive for granzyme B and perforin. In additional embodiment, CD4+ T cells may be further sorted into subpopulations. For example, CD4+ T helper cells may be sorted into naive, central memory, and effector cells by identifying cell populations that have cell surface antigens.

In one embodiment, the T cell preparations described herewith may be used for engineered Autologous Cell Therapy. The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for certain tumor antigen linked to an intracellular signaling part comprising a costimulatory domain and an activating domain.

In some embodiments, the donor T cells for use in the T cell therapy are obtained from the patient (e.g., for an autologous T cell therapy). In other embodiments, the donor T cells for use in the T cell therapy are obtained from a subject that is not the patient. The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells. In some embodiments, the therapeutically effective amount of the CAR-positive viable T cells is between about 0.4×10⁸ and about 2×10⁸ CAR-positive viable T cells. In some embodiments, the therapeutically effective amount of the CAR-positive viable T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ CAR-positive viable T cells.

Various aspects of the application are described in further detail in the following subsections.

### Red Blood Cell (RBC) Protein Coating Description

In one aspect, RBCs are isolated from human whole blood and, using a biotin reagent such as biotin-x-nhs, RBCs are coated with varying densities of biotin to provide for RBCs with different intrinsic binding capacity to streptavidin. Once RBCs are biotin coated, they are then coated with a saturating amount of streptavidin to ensure all free biotin sites on the surface of the RBC are bound to streptavidin. In a further aspect, a tagged antigen, such as avi-tagged human CD19 protein, is contacted with the streptavidin coated RBC. The surface density of the antigen affixed to RBCs can be modulated by the concentration of avi-tagged antigen which the biotin/streptavidin coated RBCs are contacted with and/or the biotin concentration used to coat the RBCs with biotin prior to the streptavidin coating. In certain aspects, a high concentration of avi-tagged antigen is contacted with the biotin/streptavidin coated RBCs to provide for maximized RBC surface antigen display, as a factor of the streptavidin density on the RBC surface. RBCs displaying varying amounts of antigen on their surface may be quantified using quantibrite PE beads and/or staining the RBCs with fluorophore conjugated antibody specific to the antigen.

Once RBCs are affixed with an antigen to an antigen density of choice, e.g. CD19 protein, and quantified, they may be counted using a method such as bright field image analysis, e.g. a VICELL™ instrument. After the antigen targeting T-cells and antigen coated RBCs are counted, they are put together, in a specified ratio, e.g. 1:1, in cell media for a duration time which includes, overnight or multi-day co-culture.

One aspect of the methods disclosed herein is that the level of antigen expression can be closely controlled. By contrast, with prior methods it has been difficult to control antigen expression levels displayed on target cell co-culture which can require months to years of work screening single cell clones of target cells with the hopes of getting differential antigen expression. With the presently disclosed methods, for example by modulating the concentration of avi-tagged antigen which biotin/streptavidin coated RBCs are contacted with and/or by modulating the concentration of biotin which the RBCs are initially contacted with, antigen affixed to the surface of the RBCs can be closely controlled. Such modulation of levels of antigen display can provide insights into lymphocytes (e.g. CAR T-cells) by, for example, reducing the level of antigen displayed can provide for determination of antigen detection limits of CAR T-cells.

In certain aspects, after co-culture, supernatant is removed and utilized for analysis, e.g. a cytokine release analysis. An important aspect of the methods disclosed herein is that by contacting lymphocytes with the antigen displaying RBCs, any cytokine release can be understood to be from the lymphocytes because RBCs do not have cytokine release capacity. In further aspects, co-cultured lymphocytes are analyzed for antigen recognition response, e.g. cell surface activation markers and/or cell surface differentiation markers. In certain aspects, after contacting with antigen displaying RBCs, the lymphocytes are washed and stained for flow cytometry-based analysis of activation marker and/or differentiation marker upregulation. In certain further aspects, the lymphocytes can be analyzed for other properties reflecting antigen recognition or lack of antigen recognition.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### EXAMPLES

### Example 1: Isolation of Red Blood Cells

Red blood cells (RBCs) were isolated from human whole blood and then biotinylated using biotin-x-nhs. To isolate RBCs, 15 mL of room temperature FICOLL™ was transferred into a 50 mL conical tube. A 10 mL layer of whole blood was then added on top of the FICOLL™, being careful not to mix the layers. The conical tube was then centrifuged for 5 minutes at 1000 rpm at room temperature, with no breaks. Next, the supernatant was removed carefully so as not to mix RBCs into the FICOLL™. The RBCs and FICOLL™ were then washed twice with 10 mL FICOLL™ directly mixed into the RBCs, and centrifuged for 5 min at 1000 rpm at room temperature, with medium break (break setting =7). Afterwards, the RBCs were washed three times with sterile PBS and then centrifuged for 5 minutes at 1000 rpm, in room temperature, with medium break. Then, the RBCs were again washed three times with sterile EAS-45 and centrifuged for 5 min at 1000 rpm, in room temperature, with medium break. Lastly, RBCs were resuspended in 10 mL of EAS-45 and placed in 4 degrees Celsius for storage.

### Example 2: Biotinylation of Red Blood Cells

Red blood cells (RBCs) were biotinylated according to the following protocol. In a sterile environment, 1.5 mL Eppendorf tubes were prepared with labels corresponding to Table 1

TABLE 1
Reagents for Biotinylation of RBCs

| | | Biotin Final Conc. (uM) | | | | | | | |
|---|---|---|---|---|---|---|---|---|---|
| | | 0.5 | 1 | 3 | 5 | 7 | 10 | 13 | 17 |
| 1x PBS (uL) | | 354 | 354 | 354 | 354 | 354 | 354 | 354 | 354 |
| Borate Buffer | | 20 | 20 | 20 | 20 | 20 | 20 | 20 | 20 |
| (uL) | | | | | | | | | |
| RBC/EAS45(uL) | | 20 | 20 | 20 | 20 | 20 | 20 | 20 | 20 |
| Biotin | 1:1 | — | — | — | — | — | — | — | — |
| (uL) | (100 mM) | | | | | | | | |
| | 1:10 | — | — | — | — | — | — | 0.51 | 0.67 |
| | (10 mM) | | | | | | | | |
| | 1:100 | — | — | 1.18 | 1.97 | 2.76 | 3.94 | — | — |
| | (1 mM) | | | | | | | | |
| | 1:1000 | 1.97 | 3.94 | — | — | — | — | — | |
| | (.1 mM) | | | | | | | | |
| | | Biotin Final Conc. (uM) | | | | | | | |
| | | 20 | 25 | 50 | 100 | 200 | 250 | 500 | 1000 |
| 1x PBS (uL) | | 354 | 354 | 354 | 354 | 354 | 354 | 354 | 354 |
| Borate Buffer | | 20 | 20 | 20 | 20 | 20 | 20 | 20 | 20 |
| (uL) | | | | | | | | | |
| RBC/EAS45(uL) | | 20 | 20 | 20 | 20 | 20 | 20 | 20 | 20 |
| Biotin | 1:1 | — | — | — | — | 0.79 | 0.99 | 1.97 | 3.94 |
| (uL) | (100 mM) | | | | | | | | |
| | 1:10 | 0.79 | 0.99 | 1.97 | 3.94 | | | | |
| | (10 mM) | | | | | | | | |
| | 1:100 | — | — | | | | | | |
| | (1 mM) | | | | | | | | |
| | 1:1000 | | | | | | | | |
| | (.1 mM) | | | | | | | | |

The labeled tubes were then filled with borate Buffer, PBS, and RBCs using the amount specified in Table 1. RBCs were allowed to settle overnight, or spun down before taking 20 uL. Once the tubes have all reagents other than biotin-x-nhs, the caps were closed and the tubes inverted a few times to create a homogenous solution. Prior to adding biotin-x-nhs, the vials of diluted biotin-x-nhs were vortexed and spun down. Then, biotin-x-nhs was added according to Table 1 above, and vortexed immediately after mixing. Afterward, the tubes were incubated on a rotator or plate shaker for 30 minutes at room temperature, then washed three times with 800 uL of EAS-45/1 % v/v BSA at 400 g for 2 min. Lastly, 200 uL of EAS-45/1% BSA was added and placed in 4 degrees Celsius for storage.

### Example 3: Coating the RBCs With Streptavidin

Equal parts of streptavidin (SA) (2 mg/mL solution in EAS-45 +2% BSA v/v) and biotinylated RBCs were placed in a 1.5 mL Eppendorf tube (i.e. 20 uL+20 uL). The mixture was incubated at room temperature for 15 min on a plate shaker modified to hold tubes. The mixture was then washed 2× with 1 mL PBS or EAS-45 with 2% v/v BSA and spun at room temp, 400g for 2 min. After washes, the RBCs were resuspended in the smallest volume EAS-45 w/ 2% BSA v/v necessary to resuspend RBCs (i.e. ˜10-30 uL).

### Example 4: Protein Coating of Streptavidin Conjugated Biotinylated RBCs

CD19 avi-tagged protein was diluted in PBS to 100 μg/mL concentration. 100 uL of the CD19 avi-tag solution was added to each population of RBCs. The mixture was resuspended thoroughly to ensure homogenous solution, and incubated at room temperature on tube compatible plate shaker for 30 min. The cells were washed. 2× with 1 mL of PBS or EAS-45 +2 % v/v BSA at 400 g for 2 min, room temperature. 100 uL of EAS-45/2% BSA was added to the cell mixture for storage in refrigerator at 4° C.

### Example 5: Contacting Anti-CD 19 CAR T-Cells with CD19 Functionalized RBCs and Measuring Effector Cell Cytokine Release

Once RBCs are coated with varying amounts of CD19 protein and quantified, they are counted using bright field image analysis using VICELL™ instrument as with the CD19 CAR T cells. Once the CD19 CAR T cells and CD19 coated RBCs are counted, they are put in a 1:1 ratio in cell media for overnight or multi-day co-culture. After co-culture supernatant is pulled for cytokine release analysis, and cells are washed and stained for flow-based analysis of activation marker upregulation. The lymphocytes can be analyzed, in addition or alternatively, for other properties, such as with flow cytometry-based assays measuring proliferation and/or differentiation.

### Example 6: Contacting Anti-GPC3 CAR T-Cells with GPC3 Functionalized RBCs and Measuring Effector Cell Cytokine Release

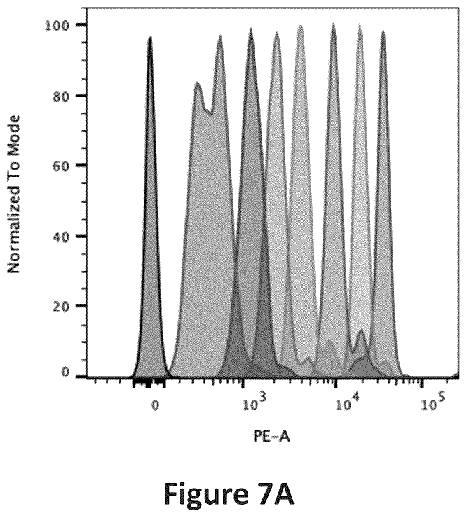

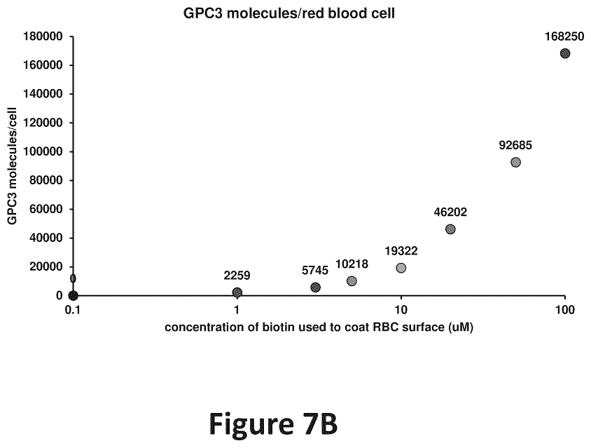

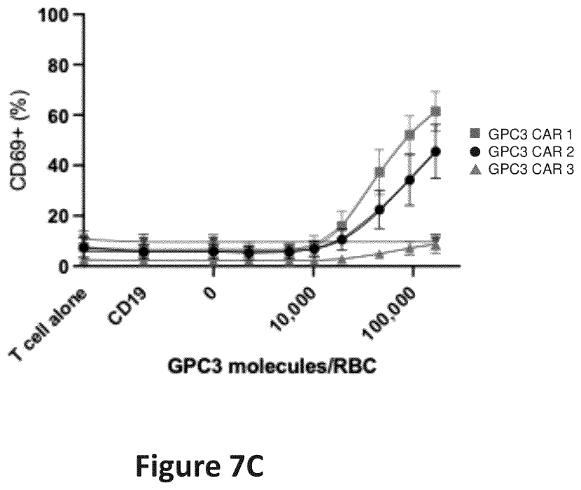

RBCs were functionalized with GPC3 as described for CD19 in Examples 1-4. Once RBCs are coated with varying amounts of GPC3 protein and quantified, they are counted using bright field image analysis using VICELL™ instrument as with the GPC3 CAR T cells. Once the GPC3 CAR T cells and GPC3 coated RBCs are counted, they are put in a 1:1 ratio in cell media for overnight or multi-day co-culture. After co-culture supernatant is pulled for cytokine release analysis, and cells are washed and stained for flow-based analysis of activation marker upregulation. The lymphocytes can be analyzed, in addition or alternatively, for other properties, such as with flow cytometry-based assays measuring proliferation and/or differentiation. Results of these assays are shown in FIG. 7A-7C .

### Example 7: Contacting Anti-BCMA Car T-Cells With BCMA Functionalized RBCs and Measuring Effector Cell Cytokine Release

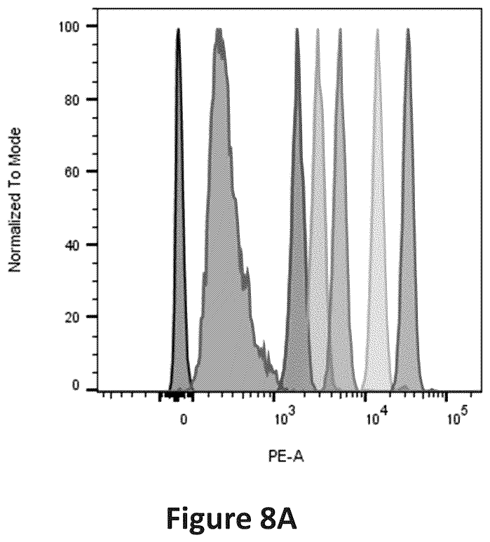

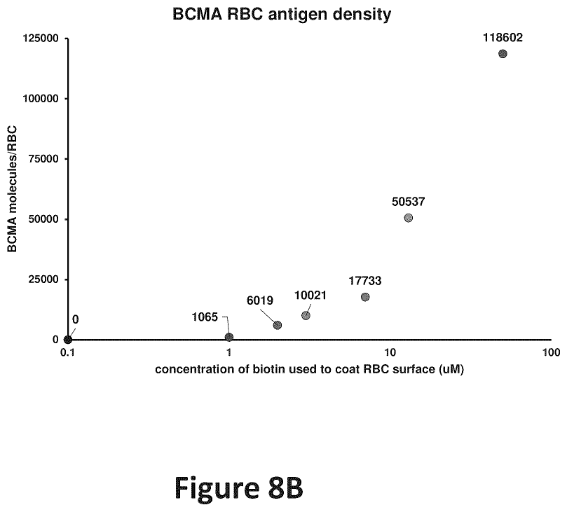

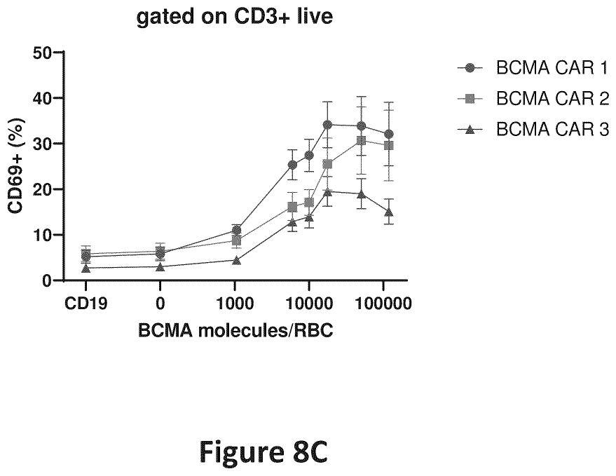

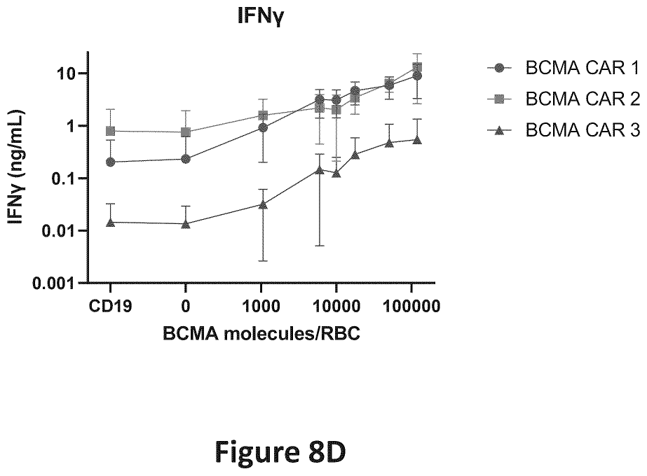

RBCs were functionalized with BCMA as described for CD19 in Examples 1-4. Once RBCs are coated with varying amounts of BCMA protein and quantified, they are counted using bright field image analysis using VICELL™ instrument as with the BCMA CAR T cells. Once the BCMA CAR T cells and BCMA coated RBCs are counted, they are put in a 1:1 ratio in cell media for overnight or multi-day co-culture. After co-culture supernatant is pulled for cytokine release analysis, and cells are washed and stained for flow-based analysis of activation marker upregulation. The lymphocytes can be analyzed, in addition or alternatively, for other properties, such as with flow cytometry-based assays measuring proliferation and/or differentiation. Results of these assays are shown in FIG. 8A-8D .

### Example 8: Two (Dual) Protein Coating of Streptavidin Conjugated Biotinylated RBCs

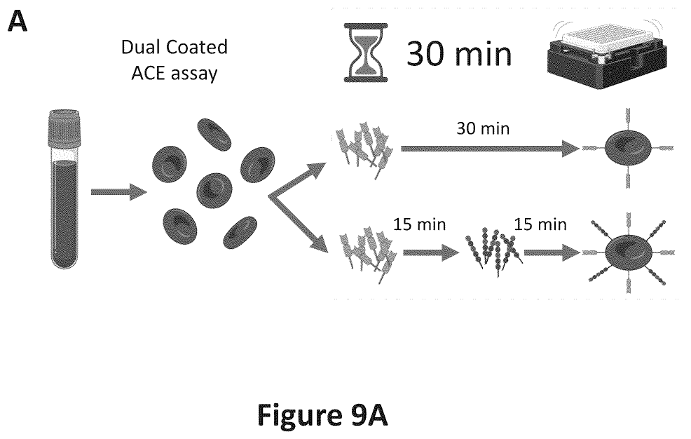

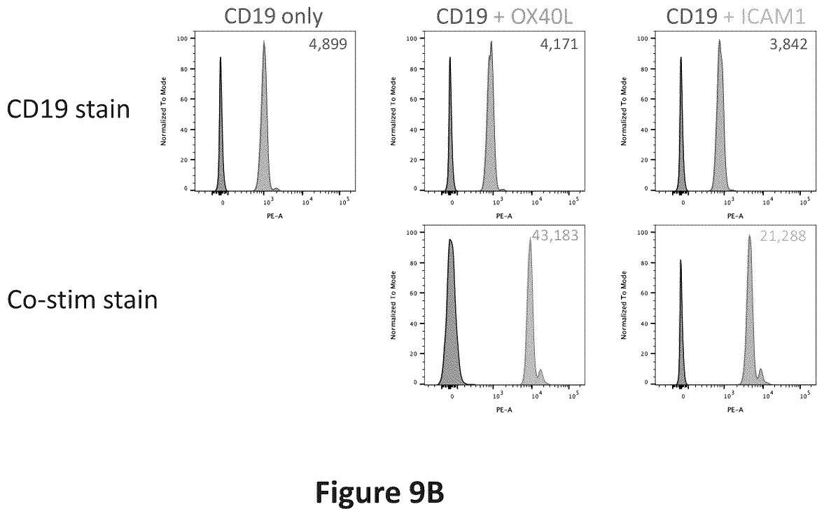

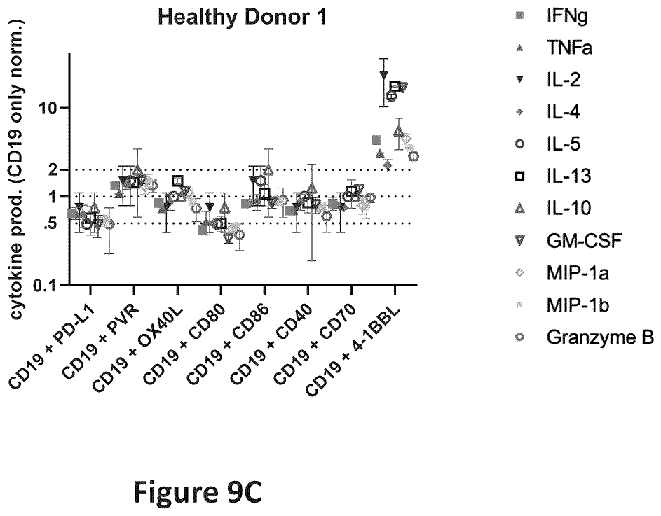

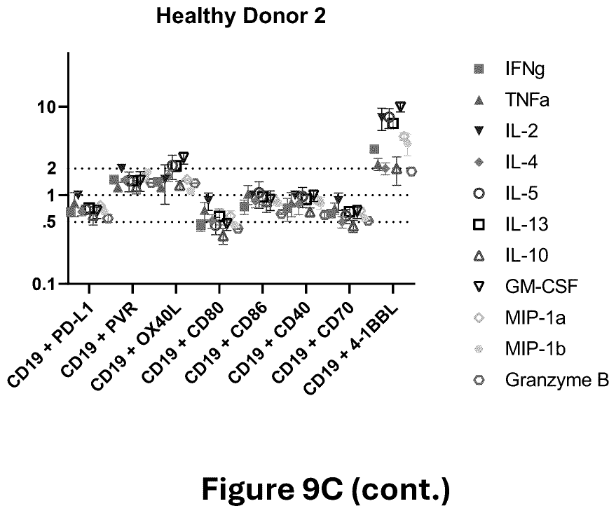

To generate CD19 single coated RBCs, CD19 avi-tagged protein was diluted in PBS to 100 μg/mL concentration. 100 uL of the CD19 avi-tag solution was added to each population of RBCs. The mixture was resuspended thoroughly to ensure homogenous solution and incubated at room temperature on tube compatible plate shaker for 30 min. For Dual coated CD19 RBCs, avi-tagged co-stimulatory ligands purchased from Acro Bio were reconstituted as suggested by the manufacturer and then further diluted in PBS to 100 μg/mL concentration. 100 uL of CD19 avi-tag solution was added to each population of RBCs for 15 minutes and then another 100 uL of co-stimulatory ligand solution was added for the final 15 minutes of incubation (30 min. incubation total for CD 19), as shown in FIG. 9A. After a total of 30 minutes of incubation the cells were washed, 2× with 1 mL of PBS or EAS-45+2% v/v BSA at 400 g for 2 min, room temperature. 100 uL of EAS-45/2% BSA was added to the cell mixture for storage in refrigerator at 4° C. Results of dual protein coating of streptavidin conjugated biotinylated beads are shown in FIG. 9B.

### Example 9: Separately Contacting Anti-CD 19 CAR T-Cells With the Functionalized Single and Dual Coated CD19 RBCs and Measuring Effector Cell Cytokine Release

Once RBCs are coated with varying amounts of CD19 protein or CD19 protein and co-stimulatory ligand and both proteins quantified, they are counted using bright field image analysis using VICELL™ instrument as with the CD19 CAR T cells. Once the CD19 CAR T cells and CD19 single or dual coated RBCs are counted, they are put in a 1:1 ratio in cell media for overnight or multi-day co-culture. After co-culture supernatant is pulled for cytokine release analysis, and cells are washed and stained for flow-based analysis of activation marker upregulation. The lymphocytes can be analyzed, in addition or alternatively, for other properties, such as with flow cytometry-based assays measuring proliferation and/or differentiation. Results of assays contacting anti-CD19 CAR T-cells with functionalized dual coated CD19 RBCs are shown for several proteins in combination with CD19 in FIG. 9C.

### Example 10: Further Assays to Which Antigen Functionalized RBCs Will Be Applied

In certain further aspects, antigen affixed RBCs can be used in methods for high throughput screening, measuring the antigen detection limit of CAR T-cells, and use in automated screening platforms. Furthermore, lymphocytes contacted with RBCs displaying target antigen can be analyzed for expression of cell surface activation and/or differentiation markers by methods, including flow cytometry.

1. A method for measuring the antigen recognition response of a population of lymphocytes wherein at least one lymphocyte of the population of lymphocytes expresses a molecule having binding specificity for an antigen comprising:
(a) affixing the antigen on a red blood cell (RBC) to create a functionalized RBC;
(b) contacting the functionalized RBC with the population of lymphocytes; and
(c) measuring for a response associated with antigen recognition exhibited by the population of lymphocytes, wherein the response is selected from the group consisting of (1) an increased cytokine release in comparison to a control population of the lymphocytes not contacted with the functionalized RBC; (2) the response is a greater proliferation of the population of lymphocytes in comparison to a control population of the lymphocytes not contacted with the functionalized RBC; (3) the response is an increased expression of cell surface activation markers of the population of lymphocytes in comparison to a control population of the lymphocytes not contacted with the functionalized RBC; (4) the response is an increased expression of cell surface differentiation markers of the population of lymphocytes in comparison to a control population of the lymphocytes not contacted with the functionalized RBC and any combination thereof.

2. The method of claim 1, wherein the population of lymphocytes comprises a cell type selected from the group consisting of a T cell, a B cell, a NK cell, a monocyte, a macrophage and any combination thereof.

3. The method of claim 2 wherein the population of lymphocytes comprises a T cell.

4. The method of claim 3, wherein the population of lymphocytes comprises a cell expressing a chimeric antigen receptor (CAR).

5. (canceled)

6. The method of claim 4, wherein the antigen is affixed to the RBC by: 1) providing a RBC, 2) biotinylating the RBC, 3) coating the biotinylated RBC with streptavidin, and 4) contacting the streptavidin-coated RBC of step 3 with a biotinylated, avi-tagged antigen.

7. The method of claim 6, wherein the RBC is contacted with a biotin solution having a biotin concentration of 0.1 μM, 0.2 M, 0.3 μM, 0.4 μM, 0.5 μM, 0.6 μM, 0.7 μM, 0.8 μM, 0.9 μM, 1 μM, 2 μM, 3 μM, 4 μM, 5 AM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 120 μM, 140 μM, 160 μM, 180 μM, 200 μM, 220 μM, 240 μM, 260 μM, 280 HM, 300 μM, 320 μM, 340 μM, 360 μM, 380 μM, 400 μM, 420 μM, 440 μM, 460 μM, 480 μM, 500 μM, 520 μM, 540 μM, 560 μM, 580 μM, 600 μM, 620 μM, 640 μM, 660 μM, 680 μM, 700 μM, 720 μM, 740 μM, 760 μM, 780 μM, 800 μM, 820 μM, 840 μM, 860 μM, 880 μM, 900 μM, 920 μM, 940 μM, 960 μM, 980 μM, or 1000 μM to provide for a biotinylated RBC.

8. The method of claim 7, wherein the biotinylated RBC is further contacted with a streptavidin solution having a streptavidin concentration of 0.5-2 mg/mL to provide for a biotin and streptavidin coated RBC.

9. The method of claim 8, wherein the biotin and streptavidin coated RBC is contacted with a biotinylated, avi-tagged antigen.

10. The method of claim 9, wherein the antigen is selected from the group consisting of DAP-10, CD 28, OX- 40, 4-1BB (CD 137), CD2, CD7, CD27, CD30, CD40, programmed death-1 (PD-1), inducible T cell costimulator (ICOS), lymphocyte function-associated antigen-1 (LFA-1, CD11a/CD18), CD3 gamma, CD3 delta, CD3 epsilon, CD247, CD276 (B7-H3), tumor necrosis factor superfamily member 14, TNFSF14, LIGHT), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, IA4, CD49D, ITGA6, VLA-6, CD49f, ICAM1, ITGAD (CD11d), ITGAE (CD103), ITGAL (CD11a), ITGAM (CD11b), ITGAX (CD11c), ITGB1, CD29, ITGB2, CD18, ITGB7, NKG2D, TNFR2, TRANCE (RANKL), DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG (Cbp), CD19a, a ligand that specifically binds with CD83, 5T4, alphafetoprotein (AFP), B-human chorionic gonadotropin, B7-1 (CD80), B7-2 (CD86), BCMA, CA-125, carcinoembryonic antigen (CEA), CD123, CD133, CD138, CD20, CD22, CD23, CD24, CD25, CD33, CD34, CD44, CD56, CD79b, CD8, CLL-1, c-Met, CMV-specific antigen, CS-1, CSPG4, CTLA-4, DLL3, disialoganglioside GD2, ductal-epithelial mucine, EB V-specific antigen, EGFR variant III (EGFR vIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, GPC3, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, Nkp30, NY-ESO-1, p53, PAP, prostase, prostate specific antigen (PSA), prostate-carcinoma tumor antigen-1 (PCTA-1), prostate-specific antigen protein, PSMA, RAGE-1, ROR1, RUI, RU2 (AS), STEAP1, STEAP2, surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20) and any combinations thereof.

11. The method of claim 10, wherein the antigen is selected from the group consisting of CD19, CD20, CD22, CD33, CLL1, GPC3, and BCMA.

12-15. (canceled)

16. The method of claim 11 wherein antigen comprises a combination of two or more antigens.

17. The method of claim 9, wherein the concentration of the antigen is selected to provide an average number of antigens affixed to an RBC between 100 antigen molecules per RBC to 500,000 antigen molecules per RBC.

18-23. (canceled)

24. The method of claim 17, wherein the functionalized RBC and the population of lymphocytes are contacted for up to 12 hours.

25-32. (canceled)

33. The method of claim wherein the cytokine is selected from the group consisting of IL-2, IFNγ, Granzyme B, GM-CSF, TNFα, Perforin, IL-4, IL-5, IL-13, IL-17, IL-10, MIP-1a, MIP-1b, IL-6, sFasL, s4-1BB, Granzyme K and any combination thereof.

34. The method of claim 1, wherein the response is an increased cytokine release in comparison to a control population of the lymphocytes not contacted with the functionalized RBC.

35. The method of claim 1, wherein the response is a greater proliferation of the population of lymphocytes in comparison to a control population of the lymphocytes not contacted with the functionalized RBC.

36. The method of claim 1, wherein the response is an increased expression of cell surface activation markers of the population of lymphocytes in comparison to a control population of the lymphocytes not contacted with the functionalized RBC.

37. The method of claim 1, wherein the response is an increased expression of cell surface differentiation markers of the population of lymphocytes in comparison to a control population of the lymphocytes not contacted with the functionalized RBC.

38. The method of claim 34, wherein the cytokines are measured in supernatant removed from the mixture of the lymphocytes and functionalized RBCs.

39. The method of claim 1, wherein the response associated with antigen recognition is measured by flow cytometric analysis of the lymphocytes after the contacting of the lymphocytes with the functionalized RBCs.

40. (canceled)

41. A method for measuring the cytokine release activity of a population of lymphocytes wherein at least one lymphocyte of the population of lymphocytes expresses a molecule having binding specificity for an antigen comprising;
(a) affixing the antigen on a red blood cell (RBC) to create a functionalized RBC;
(b) contacting the functionalized RBC with the population of lymphocytes; and
(c) measuring the cytokines released by the population of lymphocytes.
